(19) United States
(12) Patent Application Publication
(10) Pub. No.: US 2022/0409881 A1
(43) Pub. Date: Dec. 29, 2022

(54) IMPLANTABLE MEDICAL LEADS HAVING ELECTRODE SEGMENTS OF DIFFERENT SIZES

(71) Applicant: MEDTRONIC, INC., Minneapolis, MN (US)

(72) Inventors: Rene A. Molina, Maple Grove, MN (US); Michelle A. Case, Blaine, MN (US); Paula A. Dassbach, Minneapolis, MN (US); Abbey B.H. Becker, Shoreview, MN (US); Christopher L. Pulliam, Plymouth, MN (US)

(21) Appl. No.: 17/846,274

(22) Filed: Jun. 22, 2022

Related U.S. Application Data
(60) Provisional application No. 63/214,201, filed on Jun. 23, 2021.

(51) Int. Cl. A61N 1/04; A61N 1/05

(52) U.S. Cl. CPC A61N 1/0476; A61N 1/0504; A61N 1/048

(57) ABSTRACT

Implantable medical leads include rows of electrode segments where electrode segments within a given row may be a different size and/or adjacent electrode segments of adjacent rows may be of a different size. The arrangement of the electrode segments of different sizes may avoid intersections of the spaces between segments to reduce the size and/or number of blind spots that otherwise occur for delivery of stimulation signals and/or sensing of physiological signals. The electrode segments of different sizes may be of a same shape type but with different proportions.

### RELATED APPLICATIONS

This application claims priority to U.S. Provisional Application No. 63/214,201, filed Jun. 23, 2021, which is incorporated by reference herein.

### TECHNICAL FIELD

Embodiments related to implantable medical leads that include electrode segments where one or more segments of the electrode segments are different sizes than other segments.

### BACKGROUND

Implantable medical systems that include an implantable medical device and an implantable medical lead provide therapy to and/or monitoring of physiological conditions. Typically, the implantable medical device is implanted at a location of convenience which may be some distance from the target area to be stimulated or sensed. The implantable medical lead is implanted by being routed to the target area so that a position on the implantable medical lead has electrodes at the target area to deliver stimulation signals and/or sense physiological signals.

Conductors within the implantable medical lead carry electrical signals between a set of electrical connectors on a proximal end of the lead body and the electrodes located on the lead body and distal of the set of connectors. The proximal end is connected to the implantable medical device. Where the lead is not long enough to extend from the target area to the implantable medical device, a lead extension may be used where the proximal end of the lead connects to the distal end of the lead extension and the proximal end of the lead extension connects to the implantable medical device.

The distal electrodes are often rings that surround the entire circumference of the lead body. While such rings are effective in many cases, some situations call for a more precise location of the electrodes so as to steer stimulation current to and/or to capture physiological signals from a more precise location. In such a case, electrode segments may be used instead of a continuous ring. Conventionally, the electrode segments are electrically isolated and are uniformly positioned about the circumference of the lead and are uniformly sized. A given row of electrode segments are uniformly spaced about the circumference of the lead in the position where a ring would otherwise be.

These uniformly positioned and sized electrode segments provide a degree of additional precision about the circumference of the lead. However, even with the uniformly positioned and sized electrode segments, blind spots can occur at the intersections between segments of a given row and between segments of axially adjacent rows. At these blind spots, sensing of physiological signals and/or delivery of stimulation signals may be hindered. Furthermore, such uniformly positioned and sized electrodes may limit the ability to craft a desirable volume of neural activation (VNA).

### SUMMARY

Embodiments address issues such as these and others by providing leads having distal electrode segments where the segments have different sizes. For instance, within a given row, the size of the electrode segments may differ and/or the size of electrode segments in adjacent rows may differ. Furthermore, the rows of segments may be immediately adjacent, and the electrode segments may be arranged to minimize the blind spots that otherwise occur at intersections of the segments and/or to better craft the VNA.

Embodiments provide an implantable medical lead that includes a lead body having a proximal end and a distal end and a set of connectors on the proximal end. The implantable medical lead also includes a set of electrode segments on the lead body and distal of the set of connectors, the set of electrode segments being provided in at least two rows separated along a length of the lead body. A first of the at least two rows has at least two electrode segments that have a different size than each other. A second of the at least two rows is immediately adjacent to the first of the at least two rows and has at least two electrode segments that have a different size than each other. The implantable medical lead further includes a plurality of conductors with each conductor electrically connecting a corresponding connector on the proximal end to a corresponding electrode segment.

Embodiments provide an implantable medical system that includes an implantable medical device having electrical circuitry connected to electrical contacts and also includes an implantable medical lead. The implantable medical lead includes a lead body having a proximal end and a distal end, the proximal end being coupled to the implantable medical device. The implantable medical lead also includes a set of connectors on the proximal end that are coupled to corresponding electrical contacts of the implantable medical device. The implantable medical lead further includes a set of electrode segments on the lead body and distal of the set of connectors, the set of electrode segments being provided in at least two rows separated along a length of the lead body. A first of the at least two rows has at least two electrode segments that have a different size than each other. A second of the at least two rows is immediately adjacent to the first of the at least two rows and has at least two electrode segments that have a different size than each other. The implantable medical lead additionally includes a plurality of conductors with each conductor electrically connecting a corresponding connector on the proximal end to a corresponding electrode segment.

Embodiments provide a method of providing therapy to a patient that involves providing an implantable medical device having electrical circuitry connected to electrical contacts. The method further involves providing an implantable medical lead. The implantable medical lead comprises a lead body having a proximal end and a distal end, the proximal end being coupled to the implantable medical device. The implantable medical lead also includes a set of connectors on the proximal end that are coupled to corresponding electrical contacts of the implantable medical device. The implantable medical lead further includes a set of electrode segments on the lead body and distal of the set of connectors, the set of electrode segments being provided in at least two rows separated along a length of the lead body. A first of the at least two rows has at least two electrode segments that have a different size than each other. A second of the at least two rows is immediately adjacent to the first of the at least two rows and has at least two electrode segments that have a different size than each other. The implantable medical lead additionally includes a plurality of conductors with each conductor electrically connecting a corresponding connector on the proximal end to a corresponding electrode segment. The method also involves passing electrical signals between at least one of the electrode segments of the first row and at least one of the electrode segments of the second row and the electrical circuitry.

Embodiments provide an implantable medical lead that includes a lead body having a proximal end and a distal end and a set of connectors on the proximal end. The implantable medical lead also includes a set of electrode segments on the lead body and distal of the set of connectors, the set of electrode segments being provided in at least two rows separated along a length of the lead body, the set of electrode segments having at least two electrode segments that have a same shape type and different proportions than each other. The implantable medical lead further includes a plurality of conductors with each conductor electrically connecting a corresponding connector on the proximal end to a corresponding electrode segment.

### DESCRIPTION OF THE DRAWINGS

FIG. 1 shows an example of an implantable medical system that includes an implantable medical device and an embodiment of an implantable medical lead.

FIG. 2 shows an example of a proximal end of the implantable lead that is coupled to the implantable medical device or the lead extension.

FIG. 3 shows a first example of a distal end of the implantable lead with a configuration of electrode segments.

FIG. 4 shows the first example of FIG. 3 in an unrolled representation to better illustrate the configuration of the electrode segments about the circumference of the lead body.

FIG. 5 shows a second distal end example in an unrolled representation to better illustrate the configuration of the electrode segments about the circumference of the lead body.

FIG. 6 shows a third distal end example in an unrolled representation to better illustrate the configuration of the electrode segments about the circumference of the lead body.

### DETAILED DESCRIPTION

Embodiments provide implantable medical leads that have one or more rows of electrode segments where the electrode segments have a different size. Electrode segments within a given row at a particular axial position may have different sizes than one another. Electrodes segments within adjacent rows at different axial positions may have different sizes than one another. Embodiments may further provide that the electrode segments of a given row and/or in adjacent rows have a same shape type but different proportions to achieve the difference in size.

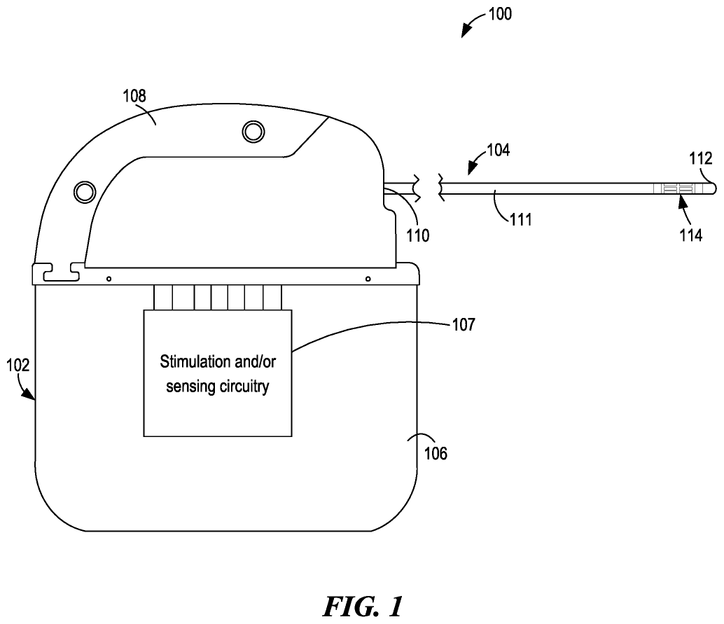

FIG. 1 shows an example of an implantable medical system 100 that includes an implantable medical device 102 and an implantable medical lead 104. The implantable medical system 100 may be of any type such as a neuromodulation stimulation and/or sensing system, a cardiac stimulation and/or sensing system, and the like. The implantable medical lead 104 and/or lead extension includes a proximal end that is installed into a lead passageway 110 of a header 108 of the implantable medical device 102. The header 108 is a housing for electrical contacts and is installed on a separately sealed housing 106 of the implantable medical device 102 that includes stimulation and/or sensing electrical circuitry 107.

As shown in subsequent figures and discussed below, the proximal end of the lead 104 and/or lead extension has electrical connectors attached to a lead body 111 of the lead 104 which may be constructed of a non-conductive biocompatible material such as polyurethane. These electrical connectors engage electrical contacts within the header 108 that are electrically coupled through a feedthrough assembly from the header 108 and to the electrical circuitry 107. Conductors within the lead body 111 of the lead 104 then carry the electrical signals between the circuitry 107 of the housing 106 and electrodes 114, including rows of electrode segments, on a distal end 112 of the lead body 111 of the lead 104. The distal end 112 is positioned at the target stimulation and/or sensing site within the body of the patient. It will be appreciated that the electrodes and electrode segments may be positioned along the lead body at any point distal of the set of proximal connectors and the positioning of the electrodes and electrode segments are shown at the distal end of the lead in the various figures herein for purposes of example.

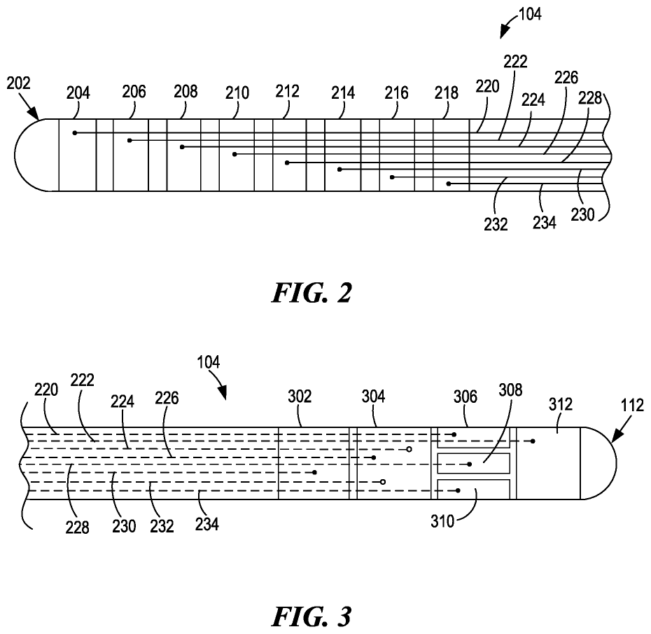

FIG. 2 shows an example of a proximal end 202 of the implantable medical lead 104 and/or lead extension. The proximal end 202 includes several proximal connectors which in this example includes eight proximal connectors 204, 206, 208, 210, 212, 214, 216 and 218 that are spaced apart from each other along the proximal end 202 to provide electrical isolation. The proximal end 202 is inserted into the lead passageway 110 of the implantable medical device 102 so that electrical connectors 204, 206, 208 and so on located on the proximal end 202 can engage electrical contacts of the implantable medical device 102. Each of the proximal connectors 204, 206, 208, and so on have an associated electrical conductor to electrically interconnect the proximal connectors to distal electrodes.

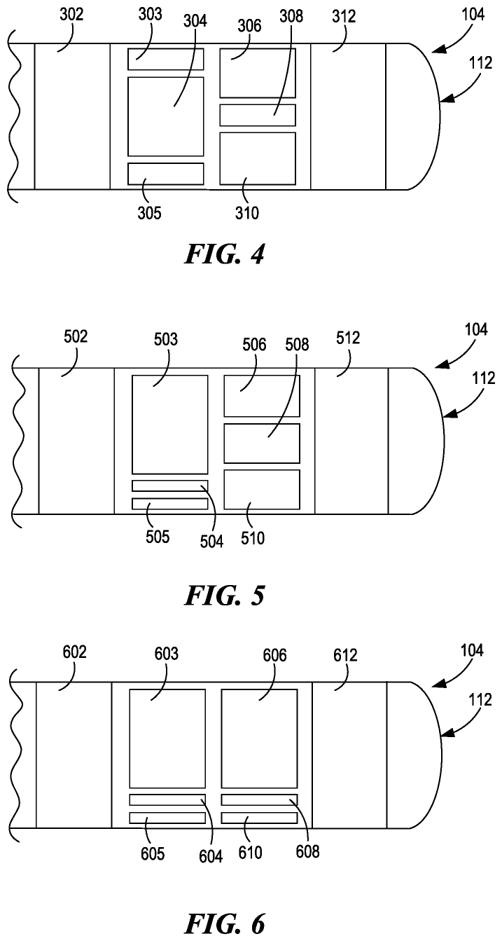

FIG. 3 shows an example of a distal end 112 of the implantable medical lead 104. The distal end includes several distal electrodes which in this case includes a mix of both non-segmented ring electrodes 302 and 312 as well as rows of electrode segments. In this particular example, the ring electrode 302 is proximal of the set of electrode segments and the ring electrode 312 is distal of the set of electrode segments. A first row of electrode segments immediately adjacent and spaced axially along a length of the lead body 111 from the ring electrode 302 includes electrode segment 304 as well as other electrode segments on the opposite side of the lead 104 that are not visible in FIG. 3 but are shown and described below in FIG. 4 as electrode segments 303 and 305. A second row of electrode segments immediately adjacent and spaced axially along a length of the lead body 111 from the first row of electrode segments includes electrode segments 306, 308, and 310 where electrode segments 306 and 310 wrap around to the backside of the lead 104. These electrode segments of each row are spaced circumferentially from each other as shown to provide electrical isolation. The ring electrode 312 is immediately adjacent and spaced axially along a length of the lead body 111 from the second row of electrode segments.

The distal end 112 is positioned at the target area within the body of the patient so that electrodes 302, 304, 306 and so on located on the distal end 112 can engage body tissue to either deliver electrical stimulation therapy pulses through the tissue or to sense electrical physiological signals emanating from the tissue. Each of the distal electrodes 302, 312 and electrode segments 303, 304, 305, 306, 308, and 310 have an associated electrical conductor attached thereto electrically interconnect the proximal connectors to distal electrodes. The electrical conductors are shown in dashed line format to preserve clarity of the electrodes and electrode segments. It will be appreciated that conductor 224 is connected to electrode segment 303 present on the back side of the lead 104 while conductor 232 is connected to electrode segment 305 also present on the backside of the lead 104.

The conductors may be constructed of electrically conductive material used in conventional leads such as various metals such as platinum, platinum-iridium alloys, carbon, and the like. The proximal connectors, distal electrodes, and distal electrode segments may be constructed of conductive materials used in conventional leads such as those listed above for the conductors. These conductors may be individually insulated such as by having a non-conductive coating or other insulator on the conductors 210, 212, 216 to avoid short circuits between conductors. The insulative coating or insulation may be of materials used in conventional leads such as polytetrafluoroethylene based materials. Because these conductors are individually insulated, they may co-exist within the lead 104, such as within a stylet lumen where contact between the conductors may occur. Furthermore, while the conductors of FIGS. 2 and 3 are shown to be linear, one or more conductors may have other configurations such as a coil shape.

FIG. 4 shows an unrolled and flattened representation of the cylindrical lead example of FIG. 3 so that the complete circumference can be seen in the plane of the page. In this example, it can be seen that the ring electrodes 302 and 312 extend from edge to edge as these ring electrodes 302 312 are continuous about the entire circumference of the lead 104. The first row of electrode segments shows that the electrode segment 304 extends over more than half of the circumference of the lead 104 but in the remaining space two additional electrode segments 303 and 305 are present with a small space between electrode segments 303 and 305 and between each electrode segment 303, 305 and electrode segment 304. As can be seen, electrode segments 303 and 305 are a different size than electrode segment 304 but all have a same shape type as rectangles, albeit electrode segments 303 and 305 are rectangles of different proportions than those of the rectangle formed by electrode segment 304. In this example, electrode segments 303 and 305 have a different circumferential size but a same axial size as the electrode segment 304.

FIG. 4 also shows the second row of electrode segments that is immediately adjacent to the first row of electrode segments. In the second row of electrode segments, the electrode segment 308 extends over only a small amount of the circumference of the lead 104 while electrode segments 306 and 310 extend over the remaining circumference with a small space between electrode segments 306 and 310 and between each electrode segment 306, 310 and electrode segment 308. As can be seen, electrode segments 306 and 310 are a different size than electrode segment 308 but all have a same shape type as rectangles in this unrolled and flattened representation, albeit electrode segments 306 and 310 are rectangles of different proportions than those of the rectangle formed by electrode segment 308. In this example, electrode segments 306 and 310 have a different circumferential size but a same axial size as the electrode segment 308.

Additionally, it can be seen that due to the differences in size in one or both of the rows of electrode segments, the space between adjacent electrode segments of a given row do not align with the space between adjacent electrode segments of the immediately adjacent row. The lack of alignment occurs because the space between electrode segments of one row is located at a different circumferential position than the space between electrode segments of the immediately adjacent row. For instance, the space between electrode segment 304 and electrode segment 305 does not align with the space between electrode segment 308 and electrode segment 310. Similarly, the space between electrode segment 303 and electrode segment 304 does not align with the space between electrode segment 306 and electrode segment 308.

This lack of alignment helps to reduce the blind spots that otherwise occur in leads with same sized electrode segments where sensing or stimulation is being performed by an electrode segment of each row, especially relative to conventional leads where the gaps between same sized electrode segments are aligned at the same circumferential position. For instance, electrode segments 303 and 304 of the first row may be electrically connected to the stimulation circuitry to provide a channel of bipolar stimulation to pass electrical stimulation signals between the stimulation circuitry and the electrode segments 303 and 304 providing a desired VNA. Meanwhile in this example, electrode segments 306, 308 and 310 of the second row may be electrically connected to sensing circuitry along with electrode 302 to provide three channels of physiological sensing to detect evoked potentials and the like from the desired VNA and to pass sensed electrical signals between these electrode segments and the sensing circuitry. It will be appreciated that many configurations of stimulation and/or sensing are possible, and since the electrodes and electrode segments are individually wired to the circuitry 107, the circuitry 107 may utilize any given electrode or electrode segment for stimulation, sensing, or both. For instance, a given bipolar stimulation channel may involve electrodes and electrode segments from multiple rows, for instance segment 303 and segment 306, depending upon the desired VNA.

Likewise, differences in size between an electrode of one row and an adjacent electrode of the next row contributes to the lack of alignment of inter-segment spaces to help reduce blind spots in this example. For instance, electrode segment 305 is adjacent to electrode segment 310 and the two have different sizes while having the same shape type but with different proportions. The same is true for electrode segment 304 relative to electrode segment 310 and for electrode segment 303 relative to electrode segment 306.

This example also provides the ability during stimulation to more precisely control the desired VNA. For instance, a more narrow VNA may be achieved than would otherwise be possible without commonly sized electrode segments within the rows.

FIG. 5 shows an unrolled and flattened representation of a second cylindrical lead example so that the complete circumference can be seen in the plane of the page. In this example, it can be seen that the ring electrodes 502 and 512 extend from edge to edge as these ring electrodes 502, 512 are continuous about the entire circumference of the lead 104. A first row of electrode segments shows that an electrode segment 503 extends over more than half of the circumference of the lead 104 but in the remaining space two additional electrode segments 504 and 505 are present with a small space between electrode segments 504 and 505 and between each electrode segment 504, 505 and electrode segment 503. As can be seen, electrode segments 504 and 505 are a different size than electrode segment 503 but all have a same shape type as rectangles, albeit electrode segments 504 and 505 are rectangles of different proportions than those of the rectangle formed by electrode segment 503. Electrode segments 504 and 505 have a different circumferential size but a same axial size as the electrode segment 503.

FIG. 5 also shows the second row of electrode segments that is immediately adjacent to the first row of electrode segments. In the second row of electrode segments, each electrode segment 506, 508, and 510 extends over only a small amount of the circumference of the lead 104 with a small space between electrode segments 506 and 510 and between each electrode segment 506, 510 and electrode segment 508. As can be seen, all electrode segments 506, 508, and 510 are a same size where all have a same shape type as rectangles of the same proportions.

Additionally, it can be seen that due to the differences in size in the first row of electrode segments, the space between adjacent electrode segments of the first row do not align with the space between adjacent electrode segments of the immediately adjacent row. For instance, the space between electrode segment 504 and electrode segment 505 does not align with the space between electrode segment 508 and electrode segment 510. As another observation, the space between electrode segments 506 and 508 does not align with the space on either side of electrode segment 503. As previously discussed, this helps to reduce the blind spots that otherwise occur in leads with only same sized electrode segments, especially where the gaps between those same sized electrode segments are aligned.

Likewise, differences in size between an electrode of one row and an adjacent electrode of the next row contributes to the lack of alignment of inter-segment spaces to help reduce blind spots in this example, even where one of the rows has same sized electrode segments as in the second row of FIG. 5. As another observation, electrode segments 506 and 508 are both adjacent to electrode segment 503 but have a different size than electrode segment 503 while having the same shape type but with different proportions.

This example also provides the ability during stimulation to more precisely control the desired VNA. For instance, as with the previous example a more narrow VNA may be achieved than would otherwise be possible without commonly sized electrode segments within the rows.

FIG. 6 shows an unrolled and flattened representation of a third cylindrical lead example so that the complete circumference can be seen in the plane of the page. In this example, it can be seen that the ring electrodes 602 and 612 extend from edge to edge as these ring electrodes 602, 612 are continuous about the entire circumference of the lead 104. A first row of electrode segments shows that an electrode segment 603 extends over more than half of the circumference of the lead 104 but in the remaining space two additional electrode segments 604 and 605 are present with a small space between electrode segments 604 and 605 and between each electrode segment 604, 605 and electrode segment 603. As can be seen, electrode segments 604 and 605 are a different size than electrode segment 603 but all have a same shape type as rectangles, albeit electrode segments 604 and 605 are rectangles of different proportions than those of the rectangle formed by electrode segment 603. Electrode segments 604 and 605 have a different circumferential size but a same axial size as the electrode segment 603.

FIG. 6 also shows the second row of electrode segments that is immediately adjacent to the first row of electrode segments. In the second row of electrode segments, it can be seen that an electrode segment 606 extends over more than half of the circumference of the lead 104 but in the remaining space two additional electrode segments 608 and 610 are present with a small space between electrode segments 608 and 610 and between each electrode segment 608, 610 and electrode segment 606. As can be seen, electrode segments 608 and 610 are a different size than electrode segment 606 but all have a same shape type as rectangles, albeit electrode segments 608 and 610 are rectangles of different proportions than those of the rectangle formed by electrode segment 606. Electrode segments 608 and 610 have a different circumferential size but a same axial size as the electrode segment 606.

Additionally, it can be seen that despite the differences in size among segments in the first row of electrode segments and the differences in size among segments in the second row of electrode segments, the size of axially adjacent electrode segments is the same. Furthermore, the space between adjacent electrode segments of the first row do align with the space between adjacent electrode segments of the immediately adjacent second row. For instance, the space between electrode segment 604 and electrode segment 605 does align with the space between electrode segment 608 and electrode segment 610. The space between electrode segments 603 and 604 does align with the space between electrode segments 606 and 608. While this configuration of electrode segments may have decreased blind spot avoidance relative to the prior examples in FIGS. 4 and 5, this example still provides the ability during stimulation to more precisely control the desired VNA. For instance, as with the previous examples a more narrow VNA may be achieved than would otherwise be possible without commonly sized electrode segments within the rows.

It will be appreciated that many variations may occur from the examples discussed above. For instance, there may be a different number of electrode segments per row than three. There may be a different number of electrode segment rows than two. The position of the rows of electrode segments relative to the ring electrodes may be different. For instance, the rings may be immediately adjacent rather than separated by the two rows of electrode segments so that one row of electrode segments is in the first or last axial position. Through each of these variations, the difference in sizes of electrode segments within the same row and/or in relation to electrode segments of an adjacent row allow for the reduction in blind spots for sensing and/or stimulation.

While embodiments have been particularly shown and described, it will be understood by those skilled in the art that various other changes in the form and details may be made therein without departing from the spirit and scope of the invention.

1. An implantable medical lead, comprising:
a lead body having a proximal end and a distal end;
a set of connectors on the proximal end;
a set of electrode segments on the lead body and distal of the set of connectors, the set of electrode segments being provided in at least two rows separated along a length of the lead body, a first of the at least two rows having at least two electrode segments that have a different size than each other, a second of the at least two rows being immediately adjacent to the first of the at least two rows and having at least two electrode segments that have a different size than each other; and
a plurality of conductors with each conductor electrically connecting a corresponding connector on the proximal end to a corresponding electrode segment.

2. The implantable medical lead of claim 1, wherein a first space is present between the at least two electrode segments of the first row and a second space is present between the at least two electrode segments of the second row, the first space occurring at a different circumferential position than the second space.

3. The implantable medical lead of claim 1, further comprising at least a third electrode segment in the first row and at least a third electrode segment in the second row.

4. The implantable medical lead of claim 3, further comprising a third space between second and third electrodes of the first row and a fourth space between second and third electrodes of the second row, the third space being at a different circumferential position than the second space and the fourth space.

5. The implantable medical lead of claim 1, further comprising a set of non-segmented electrodes on the lead body and distal of the set of connectors.

6. The implantable medical lead of claim 5, wherein a first non-segmented electrode of the set of non-segmented electrodes is proximal of the set of electrode segments.

7. The implantable medical lead of claim 6, wherein a second non-segmented electrode of the set of non-segmented electrodes is distal of the set of electrode segments.

8. The implantable medical device of claim 1, wherein the at least two electrode segments of the first row that have a different size than each other have a same shape type with different proportions.

9. The implantable medical device of claim 1, wherein the at least two electrode segments of the first row that have a different size have a different circumferential size and a same axial size.

10. An implantable medical system, comprising:
an implantable medical device having electrical circuitry connected to electrical contacts;
an implantable medical lead, comprising:
a lead body having a proximal end and a distal end, the proximal end being coupled to the implantable medical device;
a set of connectors on the proximal end that are coupled to corresponding electrical contacts of the implantable medical device;
a set of electrode segments on the lead body and distal of the set of connectors, the set of electrode segments being provided in at least two rows separated along a length of the lead body, a first of the at least two rows having at least two electrode segments that have a different size than each other, a second of the at least two rows being immediately adjacent to the first of the at least two rows and having at least two electrode segments that have a different size than each other; and
a plurality of conductors with each conductor electrically connecting a corresponding connector on the proximal end to a corresponding electrode segment.

11. The implantable medical system of claim 10, wherein a first space is present between the at least two electrode segments of the first row and a second space is present between the at least two electrode segments of the second row, the first space occurring at a different circumferential position than the second space.

12. The implantable medical system of claim 10, further comprising at least a third electrode segment in the first row and at least a third electrode segment in the second row.

13. The implantable medical system of claim 12, further comprising a third space between second and third electrodes of the first row and a fourth space between second and third electrodes of the second row, the third space being at a different circumferential position than the second space and the fourth space.

14. The implantable medical system of claim 10, further comprising a set of non-segmented electrodes on the lead body and distal of the set of connectors.

15. The implantable medical system of claim 14, wherein a first non-segmented electrode of the set of non-segmented electrodes is proximal of the set of electrode segments.

16. The implantable medical system of claim 15, wherein a second non-segmented electrode of the set of non-segmented electrodes is distal of the set of electrode segments.

17. The implantable medical system of claim 10, wherein the at least two electrode segments of the first row that have a different size than each other have a same shape type with different proportions.

18. The implantable medical system of claim 10, wherein the at least two electrode segments of the first row that have a different size have a different circumferential size and a same axial size.

19. The implantable medical system of claim 10, wherein the electrical circuitry comprises sensing circuitry and wherein at least one electrode segment of the first row and at least one electrode segment of the second row are electrically coupled to the sensing circuitry.

20-22. (canceled)

23. A method of providing therapy to a patient, comprising:
providing an implantable medical device having electrical circuitry connected to electrical contacts;
providing an implantable medical lead, comprising:
a lead body having a proximal end and a distal end, the proximal end being coupled to the implantable medical device;
a set of connectors on the proximal end that are coupled to corresponding electrical contacts of the implantable medical device;
a set of electrode segments on the lead body and distal of the set of connectors, the set of electrode segments being provided in at least two rows separated along a length of the lead body, a first of the at least two rows having at least two electrode segments that have a different size than each other, a second of the at least two rows being immediately adjacent to the first of the at least two rows and having at least two electrode segments that have a different size than each other; and
a plurality of conductors with each conductor electrically connecting a corresponding connector on the proximal end to a corresponding electrode segment; and
passing electrical signals between at least one of the electrode segments of the first row and at least one of the electrode segments of the second row and the electrical circuitry.

24-35. (canceled)
